(19) United States
(12) Patent Application Publication
(10) Pub. No.: US 2008/0200655 A1
(43) Pub. Date: Aug. 21, 2008

(54) Protein Formulations Containing Sorbitol

(75) Inventor: David C. Sek, North Andover, MA (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 12/032,478

(22) Filed: Feb. 15, 2008

Related U.S. Application Data
(60) Provisional application No. 60/901,811, filed on Feb. 16, 2007.

(51) Int. Cl. C07K 17/02

(52) U.S. Cl. 530/390.5

(57) ABSTRACT

The present invention provides a method for suppressing protein aggregation in a liquid formulation during freeze-thaw by including sorbitol in the liquid formulation. The present invention also provides methods for storing and preparing a liquid formulation containing a protein and sorbitol such that the presence of sorbitol suppresses protein aggregation during freezing and/or thawing.

### REFERENCE TO RELATED APPLICATIONS

This application claims the benefit of U.S. Provisional Patent Application No. 60/901,811, filed on Feb. 16, 2007, which is hereby incorporated by reference in its entirety.

### FIELD OF THE INVENTION

The present invention relates to methods for storing and preparing protein formulations containing sorbitol.

### BACKGROUND OF THE INVENTION

Mannitol has been generally used in protein formulations for maintaining stability and isotonicity of the formulation. In the past, liquid nitrogen has been used to quickly freeze protein formulations for storage. However, nearly all approaches to large-scale uncontrolled freezing of liquid formulations suffer from negative effects of uncontrolled solidification and melting. Inadequate control of phase change has been shown to result in product losses due to aggregation, precipitation, oxidation and denaturation. Recent technologies have been introduced to control the freeze and thaw process of protein formulations. However, these technologies typically freeze and thaw at a much slower rate. As a result, in mannitol-containing protein formulations, the slow freeze-thaw process allows crystallization of mannitol which, in turn, induces protein aggregation.

In order to avoid mannitol-induced protein aggregation during slow freeze-thaw processes, existing methods require removing mannitol from protein formulations and adding it back during post-thaw operation. These methods are expensive and require additional processing time. Therefore, there is a need for improved methods for storing and preparing protein formulations containing mannitol.

### SUMMARY OF THE INVENTION

The present invention provides an improved method for storing and preparing protein formulations. Specifically, the method of the present invention uses sorbitol in protein formulations to suppress protein aggregation during freeze and thaw. As a result, the present invention eliminates the need for removing and adding mannitol during, for example, drug product storage and filling operation. Therefore, the present invention reduces costs and processing time associated with storage and preparation of protein formulations.

In one aspect, the present invention provides a method for storing a liquid formulation including gradually freezing the liquid formulation to a temperature lower than −10° C. The liquid formulation includes a protein and sorbitol such that the presence of sorbitol suppresses protein aggregation during freezing.

In particular, the method of the present invention includes gradually freezing the liquid formulation to a temperature lower than, for instance, −20, −30, −40, −50, −70, or −80° C. In one embodiment, the temperature is at or below −30° C. In another embodiment, the temperature is at or below −40° C. In yet another embodiment, the temperature is at or below −50° C. In still another embodiment, the temperature is at or below −80° C.

Sorbitol can be present at any concentration. Typically, the sorbitol is present in a concentration no greater than approximately 4.5 M in the liquid formulation of the invention. In some embodiments, the sorbitol is in a concentration no greater than approximately 300 mM. In one particular embodiment, the concentration of sorbitol is about 300 mM. In some embodiments, the liquid formulation of the invention does not contain mannitol.

In some embodiments, the method of the present invention includes gradually freezing the liquid formulation at a rate within the range of 0.6 to 0.1° C./minute, for instance, a rate of approximately 0.6, 0.5, 0.4, 0.3, 0.2, or 0.1° C./minute.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for storing a liquid formulation of the present invention is a process intermediate.

In another aspect, the present invention provides a method for preparing a liquid formulation including gradually thawing the liquid formulation from a frozen state to a temperature higher than about 0° C. The liquid formulation contains a protein and sorbitol such that the presence of sorbitol suppresses protein aggregation during thawing.

In some embodiments, the method of the present invention includes gradually thawing the liquid formulation from a frozen state to a temperature higher than approximately 10° C., 20° C., 25° C., 30° C. or higher, for example, 37° C.

Sorbitol can be present at any concentration. Typically, the sorbitol is present in a concentration between approximately 0-4.5 M in the liquid formulation of the invention. In some embodiments, the sorbitol is in a concentration between approximately 0-300 mM. In one particular embodiment, the concentration of sorbitol is approximately 300 mM. In some embodiments, the liquid formulation of the invention does not contain mannitol.

In some embodiments, the method of the present invention includes gradually thawing the liquid formulation at a rate within the range of 0.6 to 0.1° C./minute, for instance, a rate of approximately 0.6, 0.5, 0.4, 0.3, 0.2, or 0.1° C./minute.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for preparing a liquid formulation of the present invention is a process intermediate.

In some embodiments, the liquid formulation according the invention is an aqueous formulation.

The present invention further provides a composition containing a biologically effective amount of the protein in the liquid formulation prepared by the method of the invention as described in various embodiments above.

In yet another aspect, the present invention provides a method for storing a protein in a liquid formulation including: (a) providing sorbitol to the liquid formulation; (b) gradually freezing the liquid formulation; (c) gradually thawing the liquid formulation; and wherein the presence of sorbitol suppresses protein aggregation during freeze-thaw.

Sorbitol can be present at any concentration. Typically, the sorbitol is provided to the liquid formulation to reach a final concentration no greater than approximately 4.5 M. In some embodiments, the final concentration of sorbitol is no greater than approximately 300 mM. In one particular embodiment, the final concentration of sorbitol is approximately 300 mM. In some embodiments, the liquid formulation of the invention does not contain mannitol.

In some embodiments, the gradually freezing or thawing the liquid formulation is at a rate within the range of 0.6 to 0.1° C./minute, for instance, a rate of approximately 0.6, 0.5, 0.4, 0.3, 0.2, or 0.1° C./minute.

The present invention also provides a composition containing a biologically effective amount of the protein stored in the liquid formulation by the method of the present invention as described in various embodiments above.

In still another aspect, the present invention provides a method for storing a liquid formulation including gradually cooling the liquid formulation to a temperature lower than −0° C. The liquid formulation includes a protein having a molecular weight greater than approximately 50 kDa and sorbitol such that the presence of sorbitol suppresses protein aggregation during cooling.

In some embodiments, the liquid formulation includes a protein having a molecular weight greater than approximately 75 kDa, 100 kDa, 125 kDa, 150 kDa, 175 kDa, 200 kDa, 225 kDa, 250 kDa, 275 kDa, or 300 kDa.

In some embodiments, the method of this aspect of the invention includes gradually cooling the liquid formulation to a temperature within the range of −10° C. to −80° C. or below −80° C., for instance, at or below approximately −10° C., −20° C., −30° C., −40° C., −50° C., −60° C., −70° C., or −80° C.

Sorbitol can be present at any concentration. Typically, the liquid formulation of the invention contains sorbitol in a concentration no greater than approximately 4.5 M. In some embodiments, the concentration of sorbitol is no greater than approximately 300 mM. In one particular embodiment, the concentration of sorbitol is approximately 300 mM. In some embodiments, the liquid formulation of the invention does not contain mannitol.

In some embodiments, the method of this aspect of the invention includes gradually cooling the liquid formulation at a rate within the range of 0.6 to 0.1° C./minute, for instance, a rate of approximately 0.6, 0.5, 0.4, 0.3, 0.2, or 0.1° C./minute.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for storing a liquid formulation of this aspect of the invention is a process intermediate.

In a further aspect, the present invention provides a method for suppressing aggregation of an antibody in a liquid formulation during freeze-thaw including using sorbitol instead of mannitol in the liquid formulation.

Sorbitol can be present at any concentration. Typically, the liquid formulation of the invention contains sorbitol in a concentration no greater than approximately 4.5 M. In some embodiments, the concentration of sorbitol is no greater than approximately 300 mM. In one particular embodiment, the concentration of sorbitol is approximately 300 mM.

The present invention as described above in various embodiments may be used to store and/or to prepare a liquid formulation containing a solubilized protein at any given concentration. For example, the liquid formulation may contain a protein in a concentration at or below about 35 mg/ml, 49 mg/ml, 75 mg/ml, 100 mg/ml, 125 mg/ml, 150 mg/ml, 175 mg/ml, 200 mg/ml. In other embodiments, the liquid formulation may contain a protein in a concentration greater than about 35 mg/ml, 49 mg/ml, 75 mg/ml, 100 mg/ml, 125 mg/ml, 150 mg/ml, 175 mg/ml, or 200 mg/ml.

Typically, the freezing or cooling step used in the present invention as described in various embodiments above is not accompanied by a simultaneous drying process, such as one used in a lyophilization process.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

FIG. 1 illustrates sample product temperature traces at exemplary process scales with a CryoPilot (CP) system.

FIG. 2 illustrates that mannitol induces antibody aggregation during freeze-thaw cycles.

FIG. 3 illustrates antibody aggregation in liquid formulation containing different polyols.

FIG. 4 illustrates that sorbitol suppresses aggregation of multiple exemplary proteins in liquid formulations during freeze-thaw.

FIG. 5 illustrates that sub ambient DSC scan showed that no crystallization occurred in the formulation containing sorbitol during cooling and warming.

### DETAILED DESCRIPTION OF THE INVENTION

The present invention provides improved methods for storing and preparing liquid formulations containing protein. In particular, the present invention provides a method for suppressing or eliminating protein aggregation in a liquid formulation during slow freeze and/or thaw process by including sorbitol in the liquid formulation.

Various aspects of the invention are described in further detail in the following subsections. The use of subsections is not meant to limit the invention. Each subsection may apply to any aspect of the invention.

### Protein Formulations

Proteins are relatively unstable in the aqueous state and undergo chemical and physical degradation resulting in a loss of biological activity during processing and storage. Freeze-thaw and lyophilisation are well-established methods for preserving proteins for storage. In order to preserve protein conformation, activity and stability, the protein formulations usually contain agents facilitating this, so-called lyoprotectants and cryoprotectants. Cryoprotectants are agents which provide stability to the protein from freezing-induced stresses; however, the term also includes agents that provide stability, e.g., to bulk drug formulations during storage from non-freezing-induced stresses. Lyoprotectants are agents that provide stability to the protein during water removal from the system during the drying process, presumably by maintaining the proper conformation of the protein through hydrogen bonding. Cryoprotectants can also have lyoprotectant effects. Examples of frequently used bulking agents include mannitol, glycine, sucrose, lactose, etc. The agents also contribute to the tonicity of the formulations.

As used herein, “proteins” include any recombinant or purified polypeptides including, but not limited to, antibodies, e.g., monoclonal antibodies, single chain antibodies, and other antibody variants; various growth hormones; and any pharmaceutical drug substances. Proteins referred to in this application include any naturally-occurring, modified or synthesized polypeptides.

As used herein, “a protein formulation,” “a liquid formulation,” or grammatical equivalents include any liquid polypeptide-containing compositions. The liquid polypeptide-containing compositions may further contain “buffering agent” including those agents which maintain the solution pH in an acceptable range and may include bulking agents described above and may also include histidine, phosphate, citrate, tris, diethanolamine, and the like. If the liquid polypeptide-containing compositions are pharmaceutical compositions, the liquid formulation may further contain “excipients.” The term “excipients” includes pharmaceutical acceptable carriers as well as lyoprotectants and cryoprotectants that provide proper conformation of the protein during storage so that substantial retention of biological activity and protein stability is maintained.

### Mannitol Induces Protein Aggregation During Slow Freeze and Thaw

As discussed above, freeze and thaw is a well establish method for long-term storage or as an intermediate step. However, nearly all approaches to large-scale freezing of liquid formulations suffer from negative effects of uncontrolled solidification and melting. Approaches such as freezing in bags and bottles have been repeatedly shown to result in cryoconcentration and non-uniform temperature profiles within containers. Inadequate control of phase change has been shown to result in product losses due to aggregation, precipitation, oxidation and denaturation. By contrast, controlled freeze and thaw (also referred to as slow freeze and thaw) avoids product denaturation typical of uncontrolled methods and eliminates expensive and time-consuming cleaning. In addition, overall processes benefit from a well-controlled and predictable operation.

Controlled freezing (or slow freezing) typically includes gradually freezing or cooling a liquid formulation to a temperature suitable for storage at a predetermined rate. Typically, a temperature suitable for storage includes, but is not limited to, a temperature within the range of 0° C. to −80° C. or below −80° C., preferably within the range of −10° C. to −80° C. or below −80° C., for instance, at or below about 0° C., −10° C., −20° C., −30° C., −40° C., −50° C., −60° C., −70° C., or −80° C. The gradual step down cooling can be at a rate within the range of 0.6 to 0.1° C./minute, for instance, a rate of approximately 0.6, 0.5, 0.4, 0.3, 0.2, or 0.1° C./minute.

Similarly, controlled thawing (slow thawing) typically includes gradually thawing or warming a liquid formulation to a desired temperature at a predetermined rate. In particular, the liquid formulation is thawed or warmed from a frozen state. Typically, a desired temperature for thawing purposes includes, but is not limited to, a temperature at or above about 0° C., 10° C., 20° C., or 30° C. For example, one preferred temperature is 37° C. The gradual step warming can be at a rate within the range of 0.6 to 0.1° C./minute, for instance, a rate of approximately 0.6, 0.5, 0.4, 0.3, 0.2, or 0.1° C./minute.

Controlled freeze and/or thaw may be performed in a container, such as a tube, a bag, a bottle, or any other suitable containers. The containers may be disposable. Controlled freeze and/or thaw may also be performed in a large scale or small scale. For typical large scale production, a liquid formulation may be frozen in batches of about 1 L through 300 L, for example, 1 L, 3 L, 10 L, 20 L, 50 L, 100 L, 125 L, 250 L, or 300 L. For typical small scale system, a liquid formulation may be frozen in batches of about 1 ml to 500 ml, for example, 1 ml, 10 ml, 20 ml, 30 ml, 50 ml, 100 ml, 200 ml, 300 ml, 400 ml, or 500 ml.

However, in mannitol-containing liquid formulations, the slow freezing and/or thawing allows crystallization of mannitol, which in turn, induces protein aggregation. As used herein, “protein aggregation” is meant formation of high molecular weight (HMW) species including both insoluble species detectable by turbidity measurement and soluble species detectable by size-exclusion chromatography HPLC (SEC-HPLC), cation exchange-HPLC (CEX-HPLC), X-ray diffraction (XRD), modulated differential scanning calorimetry (mDSC) and other means known to one of skill in the art.

It is observed that there is a substantial increase in the percentage of HMW species in mannitol-containing formulations upon multiple freeze and thaw cycles (see the Examples section). Increased amount of mannitol in the formulation also results in higher percentage of HMW species formation. Reduced processing volume appears to maintain the percentage of HMW species formed compared to large scale (e.g., 125 L).

An exothermal event is observed during cooling in mannitol-containing formulations. The observed enthalpy, which is due to the crystallization of mannitol as well as to the unfrozen water, increases as the processing scale increases (freeze and thaw rates decreases), or the mannitol level in the formulation increases. Crystallization event upon thawing in the mannitol-containing formulation is also observed. Without wishing to be bound by theory, the crystallization events in frozen solution suggest that the phase transition due to crystallization may induce the aggregation of protein upon freeze and thaw. Crystallization of mannitol increases with the mannitol level, which corresponds to higher % HMW formation. There was more mannitol crystallization observed in larger process scale simulation than that of the smaller scale, again correlated to greater rate of HMW formation. Decreasing mannitol in the formulation generally favors reducing HMW species formation in the liquid formulation during freeze and thaw.

### Sorbitol Suppresses Protein Aggregation

In order to address the mannitol-induced protein aggregation and to provide liquid formulations that stabilize proteins during freeze-thaw, the present invention compared the mannitol to other polyols, namely, galactitol, sorbitol, and other polyols having formula CₙH₂ₙ₊₂Oₙ. Maximum solubilities range from 180 mM to 4.5 mM depending on stereoisomers. For example, maximum solubility of galactitol is 180 mM. Maximum solubility of mannitol is 1 M. Maximum solubility of sorbitol is 4.5 M. As described in Example 2, the present invention discovered that aggregation rate is considerably faster with polyols with lower solubilities. Thus, compared to mannitol and other less soluble polyols, use of sorbitol in the liquid formulation suppresses or inhibits protein aggregation during slow freezing and/or thawing process. In particular, sorbitol may be used instead of mannitol in a liquid formulation to suppress protein aggregation during freeze-thaw.

As used herein, the terms “suppresses protein aggregation,” “inhibits protein aggregation,” or grammatical equivalents, denotes a reduction of the percentage of HMW species in a liquid formulation containing sorbitol as compared to the percentage of HMW species formed in a similar liquid formulation containing mannitol instead of sorbitol. The terms “suppresses protein aggregation” or “inhibits protein aggregation” also include eliminating formation of HMW species.

Sorbitol may be present at any concentration, limited by its maximum solubility in a given liquid formulation. Typically, sorbitol is present in a concentration no greater than approximately 4.5 M in a liquid formulation. For example, sorbitol may be in a concentration no greater than approximately 300 mM. Preferably, the concentration of sorbitol is about 300 mM.

The present invention discovered that sorbitol may be used in a variety of liquid formulations to inhibit protein aggregation during freezing and/or thawing processes. The liquid formulation may contain a solubilized protein at any given concentration. For example, the liquid formulation may contain a protein in a concentration at or below about 35 mg/ml, 49 mg/ml, 75 mg/ml, 100 mg/ml, 125 mg/ml, 150 mg/ml, 175 mg/ml, 200 mg/ml. Alternatively, the liquid formulation may contain a protein in a concentration greater than about 35 mg/ml, 49 mg/ml, 75 mg/ml, 100 mg/ml, 125 mg/ml, 150 mg/ml, 175 mg/ml, or 200 mg/ml.

Sorbitol can be used in a liquid formulation containing any protein or polypeptide as described above or known in the art. For example, the protein may be an antibody. In particular, the antibody may be a monoclonal antibody, or single chain antibody, or other antibody variants. The protein or peptide may also be a growth hormone or a pharmaceutical drug substance. The protein or polypeptide may be naturally-occurring, modified or synthesized polypeptide. The protein or polypeptide may be a small or large molecule. For example, the protein or polypeptide may have a molecular weight within the range of 25 kDa to 300 kDa or more, for instance, a molecular weight greater than approximately 25 kDa, 50 kDa, 75 kDa, 100 kDa, 125 kDa, 150 kDa, 175 kDa, 200 kDa, 225 kDa, 250 kDa, 275 kDa, or 300 kDa. The protein may be a monomer, a dimer, or a multimer.

Thus, by using sorbitol in a liquid formulation, the present invention allows slow freezing and/or thawing of the liquid formulation without inducing significant protein aggregation. The present invention is particularly useful for storing drug product containing drug substance. For example, the present invention allows all the excipients in a drug product to be present during slow freezing and/or thawing process while keeping the drug substance stable and biologically active. Therefore, the present invention eliminates the need for removing mannitol from a drug formulation before storage and adding it back during the drug product filling operation. The present invention also prevents the need for having to concentrated drug substance up to high concentrations in order to be able to add mannitol during the drug product filling operation.

Thus, the liquid formulations containing a protein and sorbitol may be stored directly in that form for later use, stored in a frozen state as an intermediate step and thawed prior to use, or subsequently prepared in a dried form, such as a lyophilized, air-dried, or spray-dried form, for later reconstitution into a liquid form or other form prior to use. In addition, compositions containing biologically active amount of the protein can be prepared and stored directly in their liquid form in accordance with the present application to take full advantage of the convenience, ease of administration without reconstitution, and ability to supply the formulation in prefilled, ready-to-use syringes or as multidose preparations if the formulation is compatible with bacteriostatic agents. The present application also provides other forms of compositions containing biologically active amount of the protein in the liquid formulation stored and prepared as described above.

It should be understood that the above-described embodiments and the following examples are given by way of illustration, not limitation. Various changes and modifications within the scope of the present invention will become apparent to those skilled in the art from the present description.

### EXAMPLES

### Example 1

### Mannitol Induces Protein Aggregation During Slow Freeze and Thaw

A formulation containing a monoclonal antibody (referred to as MAB-001 in this experiment) and 10 mM histidine, 10 mM methionine, 4% mannitol and 0.005% polysorbate-80, pH 6.0, was frozen and thawed multiple times using a CryoPilot (CP) system (Stedim Biosystems). Each freeze and thaw profile included step-down cooling to −55° C., and warming to 32° C. while the solution was mixed.

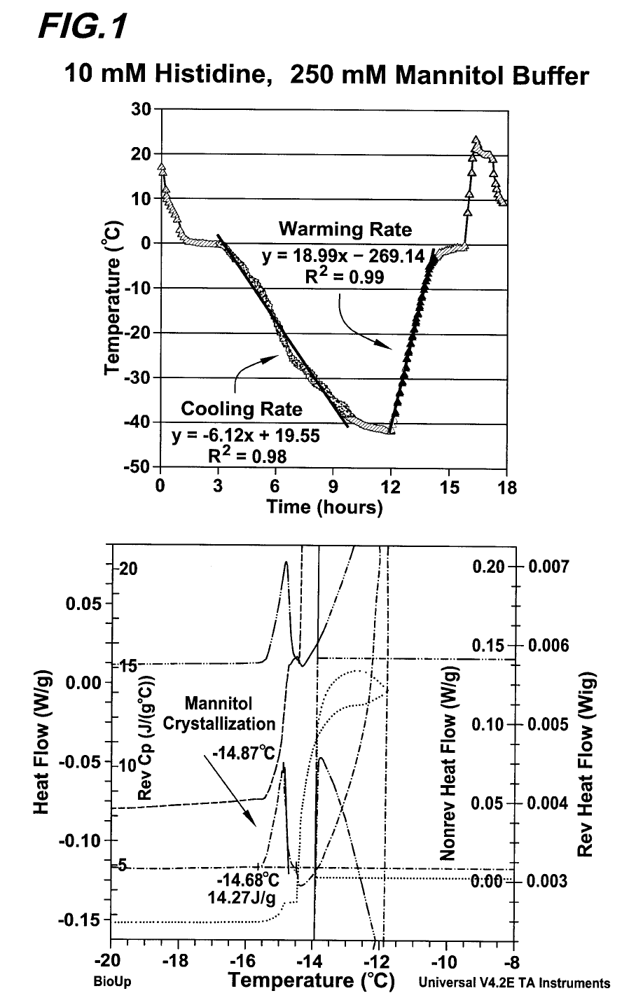

The CP simulates operation of a CryoVessel (Stedim Biosystems), the full scale production unit. The CP set point profiles for various process volumes had been developed prior to this work, to mimic behavior of the CryoVessel. FIG. 1 illustrates a sample of product temperature trace at each process scale with the CP system. Freezing (or thawing) rate was defined as the thermocouple reaching −42° C. from 0° C. (or 0° C. from −42° C.) divided by the time.

Thawed samples were analyzed primarily by SEC-HPLC and CEX-HPLC to evaluate the level of high molecular weight species (% HMW), and track the levels of acidic and basic species. Modulated differential scanning calorimetry (mDSC) and X-Ray Diffraction (XRD) were also used to assess crystallinity and polymorphs of mannitol in frozen solutions.

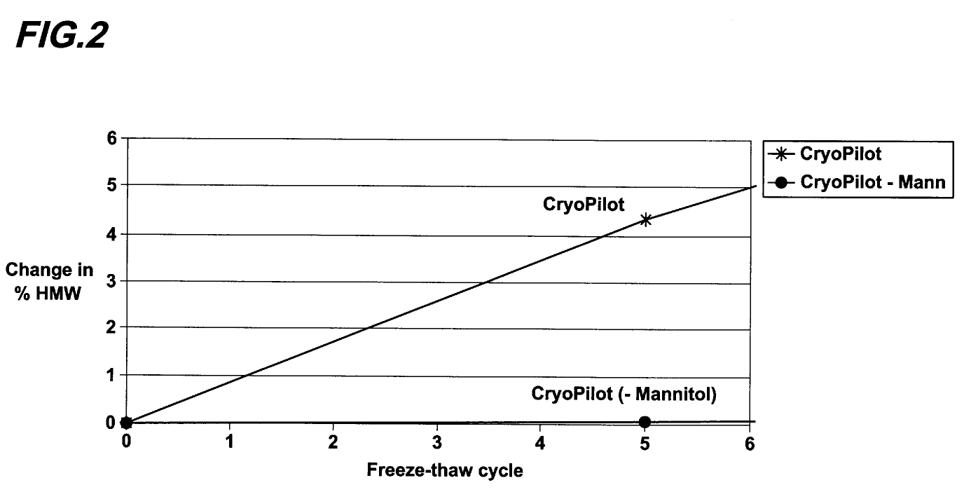

MAB-001 was found to aggregate in the presence of mannitol during slow freeze-thaw process described above. FIG. 2 illustrates formation of HMW species of MAB-001 during freeze-thaw cycles.

### Example 2

### Aggregation of Monoclonal Antibodies in Liquid Formulations Containing Different Polyols

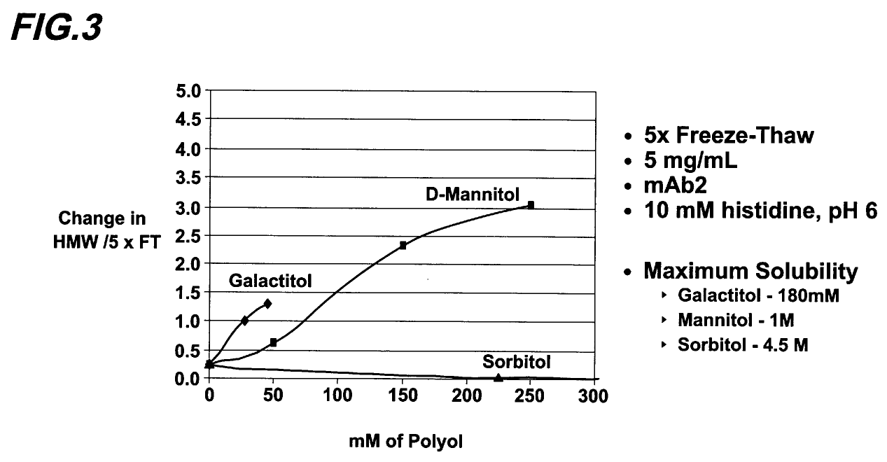

This experiment compared mannitol to other polyols with different maximum solubilities with respect to their abilities to induce aggregation during freeze-thaw. A monoclonal antibody referred to as MAB-002 was dialyzed into different liquid formulations containing mannitol, galactitol, or sorbitol, respectively, at various concentrations. Each formulation also contained 10 mM histidine, pH 6.0. The final concentration of MAB-002 in each of the liquid formulations was 5 mg/ml. Each formulation was then subject to five cycles of freeze-thaw, and monitored for HMW species formation. Changes in the percentage of HMW species was plotted against the polyol concentrations in FIG. 3. As shown in FIG. 3, galactitol, which has a maximum solubility of 180 mM, induced aggregation at low concentrations and at a faster rate compared to mannitol, while sorbitol, which has a maximum solubility of 4.5 M, did not induce aggregation significantly compared to mannitol. Therefore, this experiment shows that aggregation rate is considerably faster with polyols having lower solubilities. The liquid formulation containing sorbitol does not experience significant protein aggregation during freeze-thaw.

### Example 3

### Sorbitol Suppresses Aggregations of Multiple Proteins

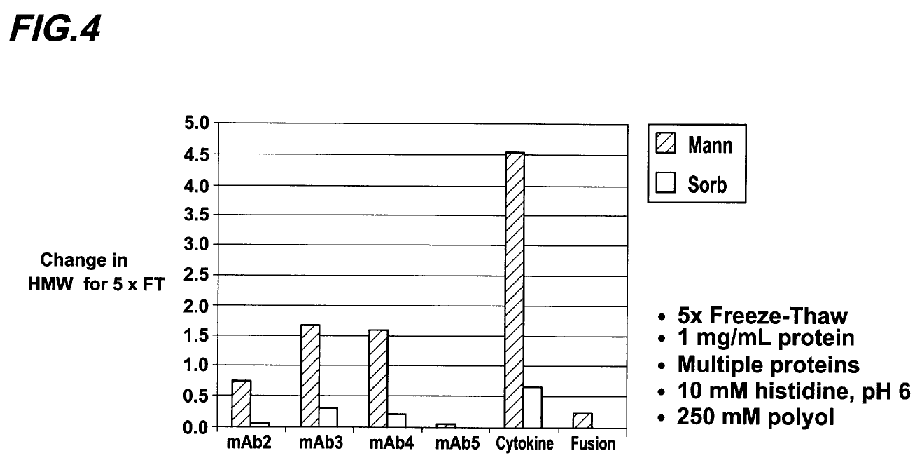

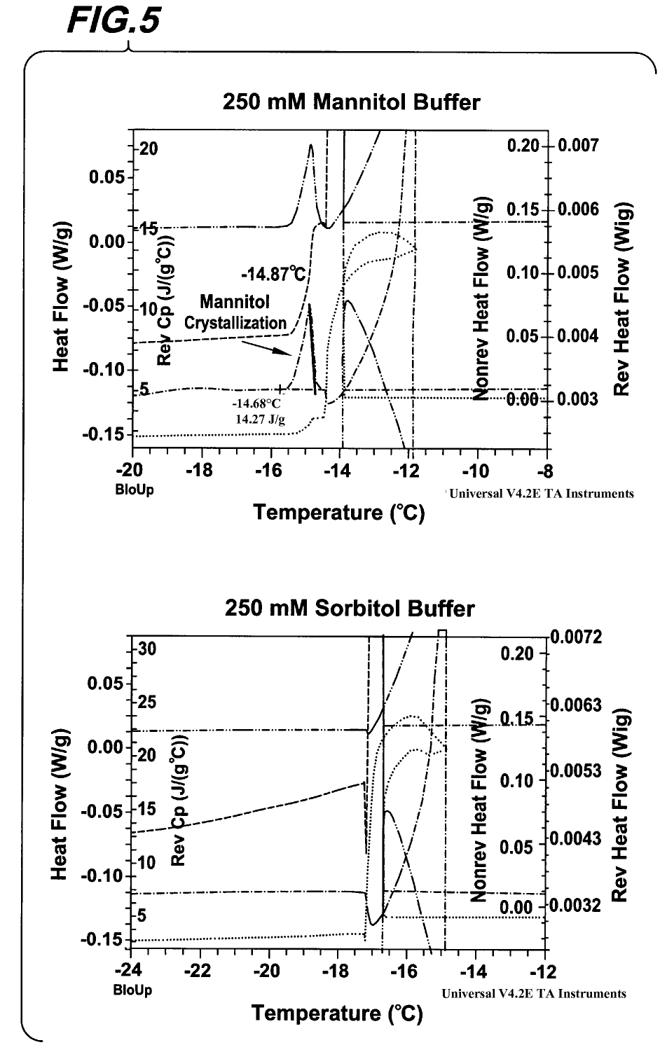

Six different proteins including four different monoclonal antibodies (referred to as mAb2, mAb3, mAb4, and mAb5), a cytokine and a fusion protein were dialyzed into 10 mM histidine, pH 6.0, and 250 mM mannitol or sorbitol. The final concentration of each protein was 1 mg/ml. The formulations were then subject to five cycles of freeze-thaw as described above, and monitored for HMW species formation. As shown in FIG. 4, formulations containing mannitol experienced considerably more protein aggregation than formulations containing sorbitol. As shown in FIG. 5, sub ambient DSC scan showed that no crystallization occurred in the formulation containing sorbitol during cooling and warming. In other words, sorbitol suppresses aggregation of multiple proteins in liquid formulations during freeze-thaw. This experiment also shows that the aggregation-suppressing effect of sorbitol is not limited to antibodies.

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### INCORPORATION BY REFERENCE

All publications and patent documents cited in this application are incorporated by reference in their entirety for all purposes to the same extent as if the contents of each individual publication or patent document were incorporated herein.

What is claimed is:

1. A method for storing a liquid formulation, the method comprising gradually freezing the liquid formulation to a temperature lower than −10° C., wherein the liquid formulation comprises a protein and sorbitol such that the presence of sorbitol suppresses protein aggregation during freezing.

2. The method of claim 1, wherein the temperature is lower than −30° C.

3. The method of claim 1, wherein the temperature is lower than −40° C.

4. The method of claim 1, wherein the temperature is lower than −50° C.

5. The method of claim 1, wherein the sorbitol is in a concentration no greater than approximately 4.5 M.

6. The method of claim 5, wherein the sorbitol is in a concentration no greater than approximately 300 mM.

7. The method of claim 1, wherein the liquid formulation does not contain mannitol.

8. The method of claim 1, wherein the freezing is at a rate of approximately 0.5° C./minute.

9. The method of claim 1, wherein the freezing is at a rate of approximately 0.3° C./minute.

10. The method of claim 1, wherein the freezing is at a rate of approximately 0.1° C./minute.

11. The method of claim 1, wherein the protein is an antibody.

12. The method of claim 11, wherein the antibody is a monoclonal antibody.

13. The method of claim 1, wherein the protein is a pharmaceutical drug substance.

14. The method of claim 1, wherein the method is a process intermediate.

15. A method for preparing a liquid formulation, the method comprising gradually thawing the liquid formulation from a frozen state to a temperature higher than 0° C., wherein the liquid formulation comprises a protein and sorbitol such that the presence of sorbitol suppresses protein aggregation during thawing.

16. The method of claim 15, wherein the temperature is higher than 20° C.

17. The method of claim 15, wherein the temperature is higher than 30° C.

18. The method of claim 15, wherein the sorbitol is in a concentration no greater than approximately 4.5 M.

19. The method of claim 18, wherein the sorbitol is in a concentration no greater than approximately 300 mM.

20. The method of claim 15, wherein the liquid formulation does not contain mannitol.

21. The method of claim 15, wherein the thawing is at a rate of approximately 0.5° C./minute.

22. The method of claim 15, wherein the thawing is at a rate of approximately 0.3° C./minute.

23. The method of claim 15, wherein the thawing is at a rate of approximately 0.1° C./minute.

24. The method of claim 15, wherein the protein is an antibody.

25. The method of claim 24, wherein the antibody is a monoclonal antibody.

26. The method of claim 15, wherein the protein is a pharmaceutical drug substance.

27. The method of claim 15, wherein the method is a process intermediate.

28. A composition comprising a biologically effective amount of the protein in the liquid formulation prepared by the method of claim 15.

29. A method for storing a protein in a liquid formulation, the method comprising:
(a) providing sorbitol to the liquid formulation;
(b) gradually freezing the liquid formulation;
(c) gradually thawing the liquid formulation; and
wherein the presence of sorbitol suppresses protein aggregation during freeze-thaw.

30. The method of claim 29, wherein the liquid formulation does not contain mannitol.

31. A composition comprising a biologically effective amount of the protein stored in the liquid formulation by the method of claim 29.

32. A method for storing a liquid formulation, the method comprising gradually cooling the liquid formulation to a temperature lower than −0° C., wherein the liquid formulation comprises a protein having a molecular weight greater than approximately 50 kDa and sorbitol such that the presence of sorbitol suppresses protein aggregation during cooling.

33. The method of claim 32, wherein the molecular weight is greater than 100 kDa.

34. The method of claim 32, wherein the molecular weight is greater than 150 kDa.

35. The method of claim 32, wherein the temperature is lower than −10° C.

36. The method of claim 32, wherein the sorbitol is in a concentration no greater than approximately 4.5 M.

37. The method of claim 32, wherein the sorbitol is in a concentration no greater than approximately 300 mM.

38. The method of claim 32, wherein the liquid formulation does not contain mannitol.

39. The method of claim 32, wherein the cooling is at a rate of approximately 0.5° C./minute.

40. The method of claim 32, wherein the cooling is at a rate of approximately 0.3° C./minute.

41. The method of claim 32, wherein the cooling is at a rate of approximately 0.1° C./minute.

42. The method of claim 32, wherein the protein is an antibody.

43. The method of claim 42, wherein the antibody is a monoclonal antibody.

44. The method of claim 32, wherein the protein is a pharmaceutical drug substance.

45. The method of claim 32, wherein the method is a process intermediate.

46. A method for suppressing aggregation of an antibody in a liquid formulation during freeze-thaw, the method comprising using sorbitol instead of mannitol in the liquid formulation.

47. The method of claim 46, wherein the sorbitol is in a concentration no greater than approximately 4.5 M.

48. The method of claim 45, wherein the sorbitol is in a concentration no greater than approximately 300 mM.
